(19) United States
(12) Patent Application Publication
(10) Pub. No.: US 2026/0272952 A1
(43) Pub. Date: Sep. 17, 2026

(54) CANNABINOIDS FOR TREATING NEUROINFLAMMATION

(71) Applicants: Lundquist Institute for Biomedical Innovation at Harbor-UCLA Medical Center, Torrance, CA (US); Robert Swift, Fort Collins, CO (US)

(72) Inventors: Michelina IACOVINO, Los Angeles, CA (US); Lynda POLGREEN, Long Beach, CA (US); Robert SWIFT, Fort Collins, CO (US)

(21) Appl. No.: 18/868,721

(22) PCT Filed: May 30, 2023
(86) PCT No.: PCT/US2023/023814

§ 371 Date: Nov. 22, 2024

Related U.S. Application Data
(60) Provisional application No. 63/346,606, filed on May 27, 2022.

(51) Int. Cl. A61K 31/00; A61P 3/00

(52) U.S. Cl. CPC A61K 31/658; A61P 3/00

(57) ABSTRACT

The present disclosure provides methods of treating a neuropathophysiological condition in a patient in need thereof. The patient can be treated with an effective amount of a cannabinoid.

### CROSS REFERENCE TO RELATED APPLICATIONS

This application claims the benefit under 35 U.S.C. § 119(e) of U.S. Provisional Application Ser. No. 63/346,606, filed May 27, 2022, the content of which is incorporated by reference in its entirety into the present disclosure.

### BACKGROUND

Mucopolysaccharidoses (MPS) is caused by the absence or malfunctioning of lysosomal enzymes needed to break down glycosaminoglycans (GAGs). These long chains of sugar carbohydrates occur within the cells that help build bone, cartilage, tendons, corneas, skin and connective tissue. GAGs (formerly called mucopolysaccharides) are also found in the fluids that lubricate joints.

Individuals with MPS either do not produce enough of one of the eleven enzymes required to break down these sugar chains into simpler molecules, or they produce enzymes that do not work properly. Over time, these GAGs collect in the cells, blood and connective tissues. The result is permanent, progressive cellular damage which affects appearance, physical abilities, organ and system functioning. Most MPS affect the central nervous system of children and result in severe progressive neurodegenerative decline eventually leading to handicap and death.

MPS III, also known as Sanfilippo syndrome, is marked by severe neurological symptoms. These include progressive dementia, aggressive behaviour, hyperactivity, seizures, some deafness and loss of vision, and an inability to sleep for more than a few hours at a time. This disorder tends to have three main stages. During the first stage, early mental and motor skill development may be somewhat delayed. Affected children show a marked decline in learning between ages 2 and 6, followed by eventual loss of language skills and loss of some or all hearing. Some children may never learn to speak. In the syndrome's second stage, aggressive behaviour, hyperactivity, profound dementia, and irregular sleep may make children difficult to manage, particularly those who retain normal physical strength. In the syndrome's last stage, children become increasingly unsteady on their feet and most are unable to walk by age 10.

No effective therapies for neurological MPS are available yet. Enzyme replacement therapies are targeted to peripheral pathology duc to inability of the recombinant enzyme to enter the brain. Gene therapy approaches are emerging; however, with limited efficacy owing to the restricted diffusion of AAV (adeno-associated virus) vectors commonly used in these protocols in the brain.

### SUMMARY

The instant disclosure provides methods for treating a neuropathophysiological condition in a patient in need thereof, comprising administering to the patient an effective amount of a cannabinoid. In certain embodiments, the neuropathophysiological condition is neuroinflammation.

The instant disclosure provides a cannabinoid for use in the treatment of a neuropathophysiological condition in a patient in need thereof. In certain embodiments, the neuropathophysiological condition is neuroinflammation.

The instant disclosure provides use of cannabinoid in the manufacture of a medicament for the treatment of a neuropathophysiological condition in a patient in need thereof. the neuropathophysiological condition is neuroinflammation.

In other aspects, the instant disclosure, in various embodiments, provides therapies for neuroinflammation associated with mucopolysaccharidoses (MPS) using cannabinoids. Such cannabinoids can be effectively delivered by intranasal, intrathecal or other routes of administrations into the brain parenchyma, where they exert a neuroprotective effect and delay or restore neuropathophysiological defects such as neuropsychiatric problems, developmental delays, mental retardation and dementia.

In some embodiments, the MPS is selected from the group consisting of MPS I, MPS II, MPS III, MPS IV, MPS VI, MPS VII, and MPS IX. In some embodiments, the MPS is MPS IIIA, MPS IIIB, MPS IIIC, or MPS IIID.

Example cannabinoids include THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabielsoin), and CBT (Cannabicitran).

In some embodiments, the cannabinoid comprises CBD. In some embodiments, the CBD is Epidiolex®.

In some embodiments, the administering is oral, transmucosal, buccal, sublingual, intranasal, intrathecal, intravenous, intramuscular, transdermal, or intraperitoneal.

In some embodiments, the CBD is administered in the form comprising lipid-based delivery system and Self-Emulsifying Drug Delivery System (SEDDS).

In some embodiments, the dose for the cannabinoid of the present disclosure is from 2.5 to 20 mg/kg per administration.

In some embodiments, the dosage form of the cannabinoids comprises about 20 mg to about 1,200 mg of a cannabinoid or a mixture of cannabinoids.

In some embodiments, the administration frequency is twice a day, once a day, once every 2 days, once every 3 days, once every 4 days, once a week, once every two weeks, once every three weeks, once a month, or once every two months.

In some embodiments, the administration dose for the cannabinoid of the present disclosure is increased or decreased per week until reaching a final dose.

In some embodiments, the administration of the cannabinoids decreases the level of the biomarkers of neuroinflammation by at least 10% in serum or in CSF in comparison with a control that received placebo treatment, without treatment, or the same patient before the cannabinoids treatment. In some embodiments, the biomarkers includes CD68, lysosomal enlargement Lamp1, IBA1, and translocator protein (TSPO), GFAP, glycosaminoglycans (GAGs), and/or heparin sulfate (HS).

In some embodiments, the administration of the cannabinoids alleviates the ncuropathophysiological condition such as dementia, aggressive behavior, hyperactivity, seizure, deafness or loss of vision by at least 10% in comparison with a control that received placebo treatment, without treatment, or the same patient before the cannabinoids treatment.

### BRIEF DESCRIPTION OF THE DRAWINGS

FIG. 1 depicts the quantification of neuropathology and storage accumulation in MPS IIIB mice: FIG. 1A shows the quantification of neuroinflammation measuring the immunoreactivity (area stained) of CD68, GFAP and Lamp1 in Carriers and Diseased MPS IIIB mice, measured 1 in 12 section along the entire rostrocaudal axis (n mice=3). FIG. 1B shows the representative images (striatum) of immunostaining for CD68, GFAP and Lamp1.

FIG. 2 depicts the impact of CBD treatment on cellular neuroinflammation. FIG. 2A shows the representative images of GFAP and CD68 immunoreactivity in mouse brain section.

FIG. 2B shows the percentage area immunoreactivity for GFAP and CD68 measured along the rostro caudal axis in a serial section. One section of every 12 of 40 μm was analyzed.

FIG. 3 depicts the GABARAPL2 accumulation decreased by CBD in forebrain neurons. HCNT: human health controls; MPS IIIA: MPS IIIA forebrain neurons.

FIG. 4 depicts the schema of Experimental Procedure. Cannabidiol treatment will start when mice are 1 month old, dosed QD. Samples are taken at 1, 3, 6, and 9 months.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment. In a preferred embodiment, the patient is human. In a more preferred embodiment, the patient is children or adolescent.

### Treatment for Mucopolysaccharidoses (MPS)

It is discovered herein that cannabinoid treatment can decrease cellular mediated neuroinflammation in MPS animal models. One embodiment of the present disclosure provides a method for treating a neuropathophysiological condition in a patient in need thereof, comprising administering to the patient an effective amount of a cannabinoid. One embodiment of the present disclosure provides a cannabinoid for use in the treatment of a neuropathophysiological condition in a patient in need thereof. One embodiment of the present disclosure provides use of cannabinoid in the manufacture of a medicament for the treatment of a neuropathophysiological condition in a patient in need thereof.

In some embodiments, the neuropathophysiological condition comprises neuroinflammation. In some embodiments, the patient being treated has a neuropathophysiological condition such as dementia, aggressive behavior, hyperactivity, seizure, deafness or loss of vision. In some embodiments, the patient has mucopolysaccharidosis (MPS).

Mucopolysaccharidoses (MPS) are caused by the absence or malfunctioning of lysosomal enzymes needed to break down glycosaminoglycans (GAGs). These long chains of sugar carbohydrates occur within the cells that help build bone, cartilage, tendons, corneas, skin and connective tissue. Seven distinct clinical types and numerous subtypes of the MPS have been identified. Examples include MPS I, MPS II, MPS III, MPS IV, MPS VI, MPS VII, and MPS IX.

MPS III, also known as Sanfilippo syndrome, is marked by severe neurological symptoms. These include progressive dementia, aggressive behavior, hyperactivity, seizures, some deafness and loss of vision, and an inability to sleep for more than a few hours at a time. This disorder tends to have three main stages in human: 1) developmental delay in cognition and language before age 3-4 years, 2) temper tantrums, hyperactivity, aggression, sleep disorder, and a further loss of cognitive and language function from age 3-4 years to age 8-10 years, and 3) loss of motor function, feeding difficulties, and seizures from age 8-10 years. Life expectancy in MPS Ill is extremely varied. Most individuals with MPS III live into their teenage years, and some live into their 20s or 30s. There are four distinct types of Sanfilippo syndrome, each caused by alteration of a different enzyme needed to completely break down the heparan sulfate sugar chain. Sanfilippo A is the most severe of the MPS III disorders and is caused by the missing or altered enzyme heparan N-sulfatase. Sanfilippo B is caused by the missing or deficient enzyme alpha-N-acetylglucosaminidase. Sanfilippo C results from the missing or altered enzyme acetyl-CoAlpha-glucosaminide acetyltransferase. Sanfilippo D is caused by the missing or deficient enzyme N-acetylglucosamine 6-sulfatase.

The term “cannabinoid” refers to a naturally occurring cannabinoid isolated from a cannabis source, or synthesized to be analogous to a naturally occurring cannabinoid isolated from a cannabis source. Synthetic cannabinoids can include cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids. The classical cannabinoids are concentrated in a viscous resin produced in structures known as glandular trichomes. At least 113 different cannabinoids have been isolated from the Cannabis plant. The main classes of cannabinoids from Cannabis are shown below in Table 1.

TABLE 1
Main classes of naturally occurring cannabinoids

| Type | Skeleton | Cyclization |
|---|---|---|
| Cannabigerol-type CBG | 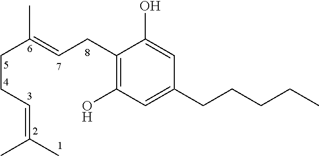 [SMILES: CCCCCc1cc(O)c(C/C=C(\C)CCC=C(C)C)c(O)c1] | 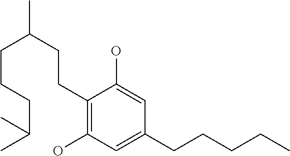 [SMILES: CCCCCc1cc(O)c(CCC(C)CCCC(C)C)c(O)c1] |
| Cannabichromene-type CBC | 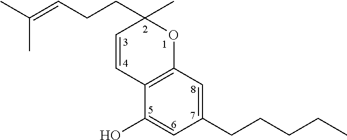 [SMILES: CCCCCc1cc(O)c2c(c1)OC(C)(CCC=C(C)C)C=C2] | 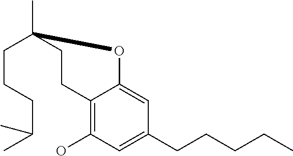 |
| Cannabidiol-type CBD | 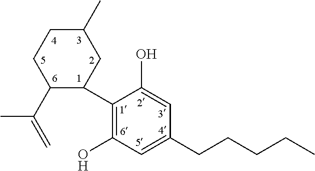 [SMILES: C=C(C)C1CCC(C)CC1c1c(O)cc(CCCCC)cc1O] | 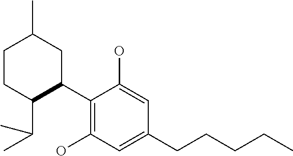 |
| Tetrahydrocannabinol- and Cannabinol-type THC, CBN | 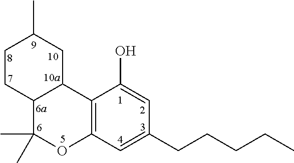 [SMILES: CCCCCc1cc(O)c2c(c1)OC(C)(C)C1CCC(C)CC21] | 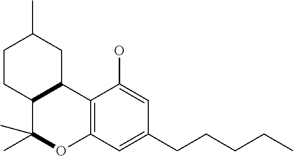 |
| Cannabielsoin-type CBE | 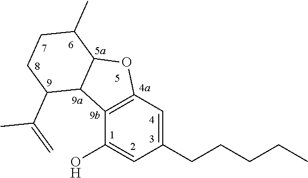 [SMILES: C=C(C)C1CCC(C)C2Oc3cc(CCCCC)cc(O)c3C12] | 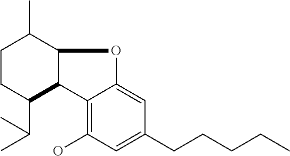 |
| iso- Tetrahydrocannabinol- type iso-THC | 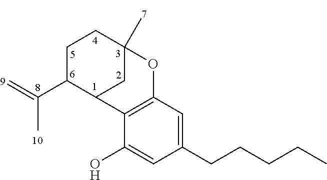 [SMILES: C=C(C)C1CCC2(C)CC1c1c(O)cc(CCCCC)cc1O2] | 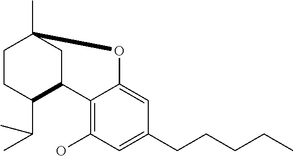 |
| Cannabicyclol-type CBL | 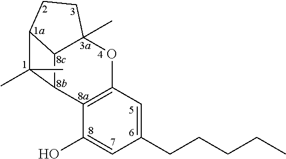 | 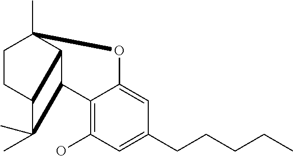 |
| Cannabicitran-type CBT | 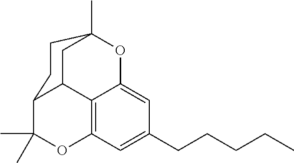 [SMILES: CCCCCc1cc2c3c(c1)OC(C)(C)C1CCC(C)(CC31)O2] | 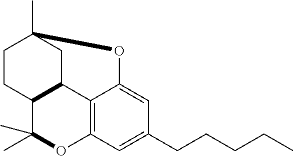 |

In certain embodiments, the cannabinoid is a natural cannabinoid. In certain embodiments, the cannabinoid is a natural cannabinoid found in a Cannabis plant. In certain embodiments, the cannabinoid is a synthetic cannabinoid. In certain embodiments, the cannabinoid is a mixture of natural cannabinoids. In certain embodiments, the cannabinoid is a mixture of synthetic cannabinoids. In certain embodiments, the cannabinoid is a mixture of natural and synthetic cannabinoids.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; and enol forms.

Representative cannabinoids include THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabiclsoin), and CBT (Cannabicitran).

The terms “cannabidiol” and “CBD” are interchangeably used herein and refer to a non-psychotropic cannabinoid having structure as described in Table 1, salt or derivatives thereof.

As a general proposition, the dosage administered to a patient of the cannabinoid of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Further, the dosage and frequency of administration of antibodies of the disclosure may be reduced by enhancing uptake and tissue penetration (e.g., into the brain) of the antibodies by modifications such as, for example, lipidation.

In some embodiments, the dose for the cannabinoid of the present disclosure is from 0.1 to 25 mg/kg per administration. In some embodiments, the dose for the cannabinoid of the present disclosure is from 2.5 to 20 mg/kg per administration. In some embodiments, the dose is at least 0.3 mg/kg, or at least 0.6 mg/kg, at least 1 mg/kg, 1.3 mg/kg, 1.6 mg/kg, 2 mg/kg, 2.5 mg/kg, 2.6 mg/kg, 3 mg/kg, 4 mg/kg, 5 mg/kg, 6 mg/kg, 7 mg/kg, 7.5 mg/kg, 8 mg/kg, 9 mg/kg, 10 mg/kg, 15 mg/kg, 20 mg/kg, or 25 mg/kg. In some embodiments, the dose is not higher than 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, or 2 mg/kg.

In some embodiments, the administration frequency is twice a day, once a day, once every 2 days, once every 3 days, once every 4 days, once a week, once every two weeks, once every three weeks, once a month, or once every two months.

In some embodiments, the administration dose for the cannabinoid of the present disclosure is increased or decreased per week, every two weeks, every four weeks, until reaching a final dose.

In some embodiments, the duration of the drug administration lasts a week, two weeks, three weeks, four weeks, two months, three months, four months, five months, six months, seven months, eight months, nine months, ten months, eleven months, twelve months, thirteen months, fourteen months, fifteen months or longer.

In some embodiments, the patient of the present disclosure has abnormal biomarker levels of neuroinflammation, such as CD68, lysosomal enlargement Lamp1, IBA1, and translocator protein (TSPO) (in microglia) and/or GFAP (in astrocyte). In some embodiments, the patient of the present disclosure has abnormal biomarker levels of the MPS, in particular MPSIII, for instance glycosaminoglycans (GAGs), such as heparin sulfate (HS).

In some embodiments, the administration of the cannabinoids decreases the serum level or cerebral spinal fluid (CSF) level of the biomarkers of neuroinflammation by about 10%, about 20%, about 30%, about 40%, about 50%, about 60%, about 70%, about 80%, or about 90%. In some embodiments, the administration of the cannabinoids decreases the serum level or CSF level of the biomarkers of MPS by about 10%, about 20%, about 30%, about 40%, about 50%, about 60%, about 70%, about 80%, or about 90% in comparison with a control. The control can be a patient without treatment, a patient that has received placebo treatment, or the same patient before cannabinoids administration.

In some embodiments, the administration of the cannabinoids alleviates the neuropathophysiological condition such as dementia, aggressive behavior, hyperactivity, seizure, deafness or loss of vision by at least 10%, at least 20%, at least 30%, at least 40%, at least 50%, at least 60%, at least 70%, at least 80%, at least 90% or 100% in comparison with a control. The control can be a patient without treatment, a patient that has received placebo treatment, or the same patient before cannabinoids administration.

A placebo is a mixture lacking any active ingredients. Generally, the placebo is made to look similar to the “real” treatment. Clinical trials are structured to comprise a drug treatment group and a placebo treatment group.

### Formulations and Methods

The present disclosure also provides pharmaceutical compositions suitable for administration, such as oral, sublingual, buccal, intranasal, intrathecal, intravenous, intramuscular, transdermal, or intraperitoneal administration. For instance, for intranasal administration, the cannabinoid and/or other agents may be retained in the submucous space of the nose, cross the arachnoid membrane, and enter into the central nervous system via the olfactory pathways. In some embodiments, a transport moiety complex is included to facilitate transport of the agent to the CNS, thereby improving response time and minimizing exposure of peripheral tissues to the active agents.

To increase the contact time and targeting to the olfactory nerves, formulation of a pharmaceutically active agent-transport moiety with a biocompatible adhesive or a delivery device can be prepared. The formulation may be in the form of a cream, liquid, spray, powder, or suppository which can be administered intranasally using a suitable applicator. Processes for preparing pharmaceuticals in these vehicles can be found throughout the literature. The formulation can be applied using any convenient method or device such as a spray device, metered dose applicator for cream, suppository suitable for intranasal insertion, and the like. The formulation can also include a bioadhesive agent, for example, a mucoadhesive agent. The mucoadhesive agent permits a close and extended contact of the composition, or the drug released from said composition, with mucosal surface by promoting adherence of said composition or drug to the mucosa. The mucoadhesive agent is preferably a polymeric compound, such as preferably, a cellulose derivative but it may be also a natural gum, alginate, pectin, or such similar polymer. A preferred cellulose derivative is hydroxypropyl methylcellulose, commercially available from Dow Chemical Co. The mucoadhesive agent can be present in from about 5 to about 25%, by weight, preferably in from about 10 to about 15% and most preferably about 10%.

Bioadhesive microparticles or nanoparticles can constitute still another component of the intranasal formulations suitable for use in the present disclosure. The bioadhesive particles include derivatives of cellulose such as hydroxypropyl cellulose and polyacrylic acid and can provide sustained release of the pharmaceutically active agents for an extended period of time (possibly days) once they are placed in the appropriate formulation. A formulation comprising bioadhesive particles can provide a multi-phase liquid or semi-solid preparation which does not seep from the nose. The microparticles or nanoparticles cling to the nasal epithelium and can release the drug over extended period of time, for example, for several hours or more.

The biocompatible adhesives can include viscosity enhancers such as methylcellulose, sodium carboxymethylcellulose, chitosan, carbopol 934P and Pluronic 127. Thermogelling agents such as ethyl (hydroxyethyl) cellulose and Pluronic 127 can also be used to advantage. Thermogelling agents are liquid at room temperature and below, but at physiological temperatures (e.g., 32-37° C.), the viscosity of the solution increases such that the solution becomes a gel.

Pharmaceutical compositions may be formulated in combination with any suitable pharmaceutical vehicle, excipient or carrier that would commonly be used in this art, such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20th Edition, A. R. Gennaro, ed., (2000). The choice of suitable carriers will depend on the exact nature of the particular vaginal dosage form desired, e.g., whether the chemotherapeutic agent and/or inhibitor of membrane efflux systems is/are to be formulated into a cream, lotion, foam, ointment, paste, solution, microemulsions, liposomal suspension, microparticles, nanoparticles or gel, as well as on the physicochemical properties of the active ingredient(s).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In certain embodiments, the pharmaceutical composition is formulated for delayed release of a cannabinoid or a mixture of cannabinoids. In certain embodiments, the pharmaceutical composition is at least partly coated by an enteric-coating agent.

In certain embodiments, the pharmaceutical composition is liquid at room temperature. In certain embodiments, the pharmaceutical composition is semi-solid at room temperature. In certain embodiments, the pharmaceutical composition is solid at room temperature.

In an embodiment, the composition is formulated in accordance with routine procedures as a pharmaceutical composition adapted for oral administration to human beings. In certain embodiments, the formulation comprises lipid-based delivery system and Self-Emulsifying Drug Delivery System (SEDDS).

In an embodiment, the composition is lipid-based delivery system. The composition may comprise water, alcohol such as ethanol, a co-solvent such as propylene glycol or polyethylene glycol, a stabilizer such as butylated hydroxyanisole (BHA) or butylated hydroxytoulene (BHT), a pharmaceutically acceptable sweetener such as sucralose, sucrose, sorbitol or fructose and an anti-oxidant e.g., propyl gallate, lecithin, Vitamin E tocopherol, sesamin, sesamol, sesamolin, alpha tocopherol, ascorbic acid, ascorbyl palmitate, fumaric acid, malic acid, and sodium mctabisulphite, disodium EDTA, and combinations of any of the foregoing.

In certain embodiments, the composition is formulated with further pharmaceutically acceptable excipients. Non-limiting examples of such pharmaceutically acceptable excipients include solubilizers for said cannabinoid, stabilizer, bases, preservatives, buffers, viscosity modifiers, bulking agents, gelling agents, emulsifiers, absorption enhancers, surfactants, etc. Further examples of ingredients can found, for example, in the United States patent or application Publication U.S. Pat. Nos. 8,222,292B2, 9,345,771B2, and US20090181080A1, hereby incorporated by reference in its entirety.

The SEDDS refers to formulations that are isotropic mixtures of oil, surfactant (with or without co-surfactant) and co-solvent which spontaneously emulsify when exposed to an aqueous medium with gentle agitation. The term “emulsifier” as used herein are amphiphilic molecules that are surface active agents and that stabilize emulsions by reducing the interfacial tension. The term “self-emulsifying” as used herein refers to a composition that forms an emulsion when placed in an aqueous medium. SEDDS have most commonly been studied to improve bioavailability of poorly water soluble drugs via oral administration. The addition of a co-solvent plays a key role in the formation of a self-emulsifying system by significantly reduces the interfacial tension. In so doing, it creates a fluid interfacial film with sufficient flexibility to take up different curvatures required to form microemulsion over a wide range of compositions.

The agents of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In one aspect, the present disclosure provides a dosage form comprising any the cannabinoids or a mixture thereof.

In certain embodiments, the dosage form comprises at least about 50 mg of a cannabinoid or a mixture of cannabinoids. In certain embodiments, the dosage form comprises about 20 mg to about 1,200 mg of a cannabinoid or a mixture of cannabinoids. In certain embodiments, the dosage form comprises about 20 mg, about 25 mg, about 50 mg, about 100 mg, about 200 mg, about 300 mg, about 400 mg, about 500 mg, about 600 mg, about 700 mg, about 800 mg, about 900 mg, about 1000 mg, about 1200 mg, about 1500 mg, or about 2000 mg of a cannabinoid or a mixture of cannabinoids.

In certain embodiments, the cannabinoid composition or dosage form comprises cannabidiol (CBD). In certain embodiments, the CBD is Epidiolex® (an oral solution containing CBD).

### EXAMPLES

### Example 1. In Vivo Study of Cannabidiol Treatment in MPS IIIA and MPS IIIB Mouse Models

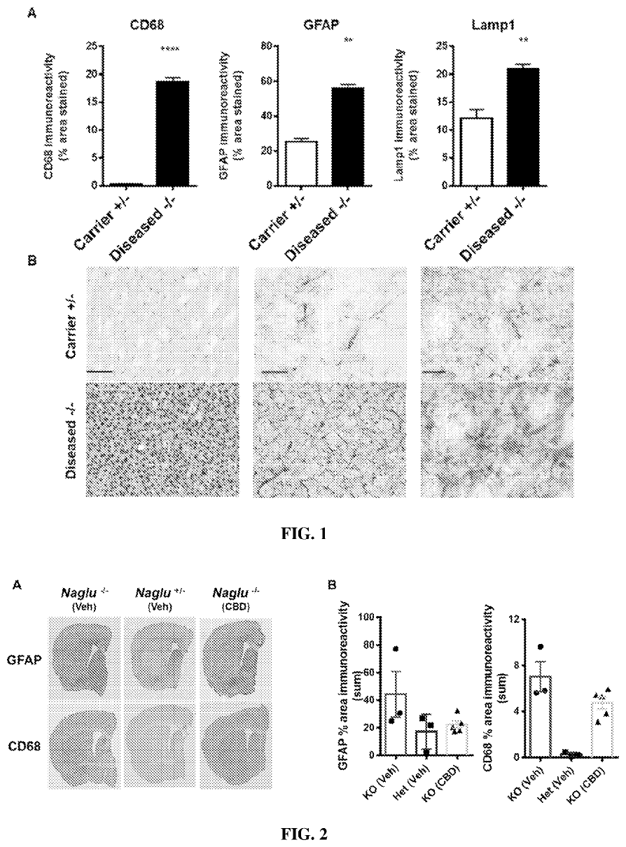

The early stage of Sanfilippo syndrome is characterized by hyperactive, aggressive behavior, and cognitive decline. These pathologies are recapitulated in both MPS IIIA and MPS IIIB mouse models. The neuroinflammatory biomarkers CD68, glial fibrillary acidic protein (GFAP) and lysosomal enlargement Lamp1, have increased immunoreactivity in MPS IIIB mice (FIG. 1).

This example performed a pilot experiment to evaluate whether CBD treatment could decrease cellular mediated neuroinflammation by quantifying microglial activation (measured as CD68 immunoreactivity) and astrocytosis (measured as glial fibrillary acidic protein (GFAP) immunoreactivity).

Sanfilippo B mice (Naglu^{-/-}) (9 months old animals) were treated with Cannabidiol (CBD purity greater than 99.9%) at 100 mg/kg. As controls, unaffected mice (Naglu+/−) and diseased mice were treated with vehicle (sesame oil 99.9% pure). Treatment was performed orally, using gavage, once/day for 5 days/week, for 4 weeks. After 4 weeks, animals were sacrificed, and the brain was collected for immunostaining. Half hemisphere was sliced using zevic slicer apparatus, and brain slices were individually fast frozen for further downstream measurements upon harvesting the brain. The other half was fixed with PFA, and 40 μM section obtained using a freezing microtome. Every 1 in 12 sections (encompassing the whole rostrocaudal axis of the brain) were immunostained using CD68 to measure microglia activation and Gfap to measure astrocytes activation. Sections were mounted on slides and imaged using Zeiss M2 microscope and MBF motorized stage. Images were quantified as percentage immunoreactivity of CD68 and Gfap using Image pro Premiere software.

### Results

CBD treatment was performed at 9 months of age when the disease was at a terminal stage. The mice were dosed daily for 30 days, with 100 mg/kg CBD in sesame oil (CBD) or sesame oil (Vehicle). FIG. 2A shows a representative image of brain section immunostained for CD68 and GFAP, markers for microglia and astrocytes activation, respectively. FIG. 2B shows the percentage staining of CD68 and Gfap, in serial section (one every twelve sections of 40 μm thick) along the rostrocaudal axis of the brain. As shown, CBD treatment decreased both microglia and astrocyte activation. Sanfilippo syndrome is a genetic, devastating, and incurable disease that impacts children. The preliminary data support the hypothesis that CBD can decrease cellular neuroinflammation on Sanfilippo B mice. Given that neuroinflammation seems to be responsible for the hyperactive behavior and learning disability associated with Sanfilippo mouse model, CBD may be an effective method for developing disease-modifying therapies for Sanfilippo syndrome.

### Example 2. In Vitro Studies of MPS IIIA with Cannabidiol Treatment

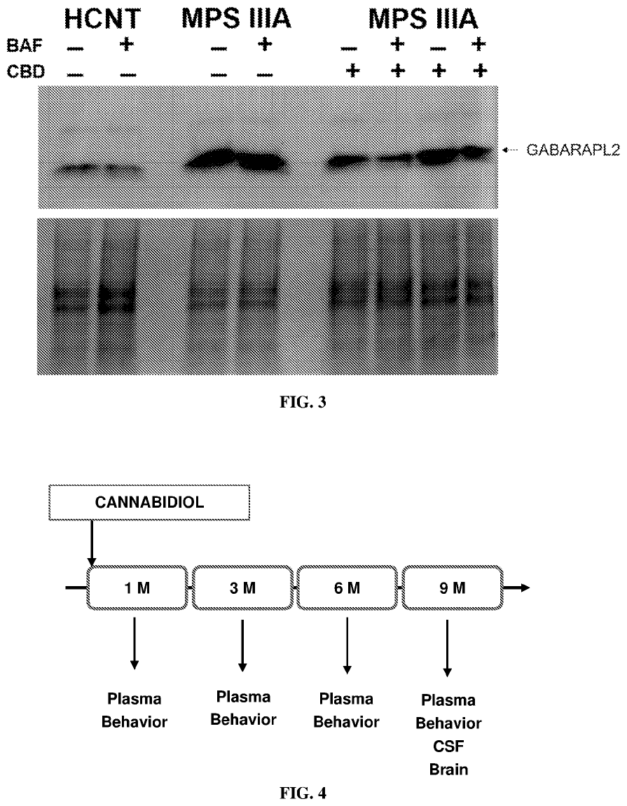

Cortical neurons differentiated from healthy control and MPS IIIA patients' iPSCs. MPS IIIA neurons were treated with CBD 1 μM (lane 5, 6) and 5 μM (lanes 7, 8), and Bafilomycin A1 200 nM (BAF, a macrolide antibiotic drug that inhibits autophagy, lanes 2, 4, 6, 8). GABARAPL2, a protein which involves in autophagy, was measured in a western blot assay (see FIG. 3). The treatment with CBD decreases the GABARAPL2 accumulation, suggesting that the CBD treatment can promote autophagy.

### Example 3. In Vivo Evaluation of Cannabidiol Effect on MPS IIIB Disease Progression

A longitudinal study will be performed with two different doses of cannabidiol to determine its effect on hyperactive, aggressive behavior, and cognitive decline in MPS IIIB mice. Mice will be treated with cannabidiol at one month of age for eight months until they reach late-stage disease. The longitudinal approach will allow evaluating the effect of cannabidiol on disease progression. FIG. 4 shows a schema of the study.

### 1. Effect of CBD on MPS IIIB Mouse Behavior and Cognitive Decline

In this example, the effect of cannabidiol on MPS IIIB mouse behavior and cognitive decline at 1, 3, 6 and 9 months will be measured, using behavioral tests to measure hyperactive and social interaction, and cognitive tests to measure spatial memory and learning deficits.

Mice will be divided into 5 groups for behavioral studies as shown in Table 2. Behavioral testing will be performed at 1, 3, 6, and 9 months at the UCLA Behavioral Testing Core. 33 mg/kg and 100 mg/kg CBD will be used in the dose-response. SHIRPA primary screen (for overall health) and rotarod testing will be performed to evaluate physical impairments that could affect the testing.

TABLE 2
Behavior Groups

| Genotype | Treatment | # |
|---|---|---|
| WT | Vehicle | 24 |
| KO | Vehicle | 24 |
| WT | CBD 30 (mg/kg) | 24 |
| WT | CBD 100 (mg/kg) | 24 |
| KO | CBD 30 (mg/kg) | 24 |
| KO | CBD 100 (mg/kg) | 24 |

WT: wild-type;
KO: Knocked-out

MODIFIED BARNE MAZE. This test is performed on a circular platform with numerous escape holes around the center of the platform. Bright overhead lighting encourages the animal to seek out the Target Escape Hole. Visual cues placed around the maze act as spatial cues. The escape latency, distance moved, and velocity are measured during the test.

NOVEL OBJECT/SOCIAL INTERACTION TASK. Children with MPS III show aberrant social interactions. The interaction of MPS IIIB mice with same-sex, non-littermate, non-cagemate mice will be measured to determine 1) the socialization of MPS IIIB mice and 2) the ability of MPS IIIB mice to recognize an object with which they have not previously interacted. Mice will also undergo a novel object recognition task with an inanimate object (e.g., hard plastic baby toy) to separate novel object recognition and socialization components of this test. The measured outcomes are time interacting with a novel mouse/object, time interacting with known mouse/object, time spent in an empty chamber, and distance moved near objects.

OPEN FIELD TEST. Children with MPS III show altered activity and anxiety-like behaviors. MPS IIIB mice show increased propensity for the center of the open field, indicating reduced fear/anxiety. This test measures exploratory behavior, anxiety, and locomotion. The measured outcomes are time in center, distance walked, rate of distance walked, and cross-overs per minute.

ELEVATED PLUS MAZE. The Elevated Plus Maze (EPM) test measures anxiety-related behavior in rodent models of CNS disorders. The apparatus consists of a “+”-shaped maze elevated above the floor with two oppositely positioned closed arms, two oppositely positioned open arms, and a center area. The animal is free to explore the maze while recorded using a video tracking system. The preference for being in open arms over closed arms (expressed as either as a percentage of entries and/or a percentage of time spent in the open arms) is calculated to measure anxiety-like behavior.

Y MAZE SPONTANEOUS ALTERNATION. Testing is performed in a Y-shaped maze with three white arms at a 120° angle from each other. The animal is introduced to the center of the maze and is allowed to explore the arms freely. Throughout multiple arm entries, the subject should show a tendency to enter a less recently visited arm. The number of arm entries and the number of triads are recorded to calculate the percentage of alternation.

Statistical methods: Intergroup comparisons for behavioral variables will be performed using multivariate ANOVA (F and p-values reported). Bonferroni corrections will be used to adjust for multiple comparisons. Behavioral results will be expressed as mean±S.E.M.

### 2. Effect of CBD in Neuroinflammation in the MPS IIIB Mice

In this experiment, cellular neuroinflammation at 6 and 9 months of age will be measured by measuring microglial activation, astrocytosis, and neuronal dysfunction. MPS IIIB mice have neuropathology as shown by activation of microglia and astrocytes. At six and nine months of age, animals will be sacrificed, the brain and spinal cord collected for neuropathology examination and immunostaining. Lamp1 expression will be measured, a lysosomal protein that is upregulated when storage accumulates due to an increase in lysosomal size. Other markers of the disease that are upregulated are glypican3 and SCMAS. Half brain hemisphere will be used for neuropathology analysis using the immunohistochemistry approach. One in every 12 brain sections (40 μm each) will be immunestained using specific antibodies for activated microglia. Microglial activation by expression of CD68, iba1, and TSPO, and astrocytosis by expression of GFAP. The accumulation of heparan sulfate and other glycosaminoglycans (GAGs) will be measured to determine if cannabidiol treatment affects the enzymatic deficiency of MPS IIIB mice. Quantification of these markers will be measured as percentage area stained throughout the rostrocaudal axis of the brain. Quantification will be performed as % of immunoreactivity using Image Pro Premiere (Media Cybernetics Inc.).

### 3. Effect of CBD on Inflammation Signaling Molecules

The effect of cannabidiol treatment on systemic and brain inflammation will be measured by measuring the change in inflammation signaling molecules, e.g. cytokines and chemokines, in plasma, CSF and brain homogenates. Inflammation signaling molecules such as caspase-1, IL-1a, IL-1β, IL-1Ra, MCP-1, KC, MIP-1, SCF-1a, and VEGF, HS, IL-1Ra, TLR4, and NLRP3 will be measured in plasma at 1, 3, 6, and 9 months and in CSF and brain homogenates at 6 and 9 months.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating a neuropathophysiological condition in a patient in need thereof, comprising administering to the patient an effective amount of a cannabinoid.

2. The method of claim 1, wherein neuropathophysiological condition comprises neuroinflammation.

3. The method of claim 1, wherein the patient has mucopolysaccharidosis (MPS).

4. The method of claim 3, wherein the mucopolysaccharidosis (MPS) is selected from the group consisting of MPS I, MPS II, MPS III, MPS IV, MPS VI, MPS VII, and MPS IX.

5. The method of claim 4, wherein the MPS is MPS IIIA, MPS IIIB, MPS IIIC, or MPS IIID.

6. The method of claim 4, wherein the MPS is MPS IIIA or MPS IIIB.

7. The method of claim 1, wherein the neuropathophysiological condition is associated with dementia, aggressive behavior, hyperactivity, seizure, deafness or loss of vision.

8. The method of claim 1, wherein the cannabinoid is selected from the group consisting of THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabielsoin), and CBT (Cannabicitran).

9. The method of claim 8, wherein the cannabinoid comprises CBD (Cannabidiol).

10. The method of claim 1, wherein the dose for the cannabinoid is from 2.5 to 20 mg/kg per administration.

11. The method of claim 9, wherein the CBD is Epidiolex®.

12. The method of claim 1, wherein the administering is oral, transmucosal, buccal, sublingual, intranasal, intrathecal, intravenous, intramuscular, transdermal, or intraperitoneal.

13. The method of claim 9, wherein the CBD is administered in the form comprising lipid-based delivery system and Self-Emulsifying Drug Delivery System (SEDDS).

14. The method of claim 1, wherein the dosage form of the cannabinoids comprises about 20 mg to about 1,200 mg of a cannabinoid or a mixture of cannabinoids.

15. The method of claim 1, wherein the administration frequency is twice a day, once a day, once every 2 days, once every 3 days, once every 4 days, once a week, once every two weeks, once every three weeks, once a month, or once every two months.

16. The method of claim 1, wherein the administration dose for the cannabinoid of the present disclosure is increased or decreased per week until reaching a final dose.

17. The method of claim 1, wherein the administration of the cannabinoids decreases the level of the biomarkers of neuroinflammation by at least 10% in serum or in CSF in comparison with a control that received placebo treatment, without treatment, or the same patient before the cannabinoids treatment.

18. The method of claim 17, wherein the biomarkers includes CD68, lysosomal enlargement Lamp1, IBA1, and translocator protein (TSPO), GFAP, glycosaminoglycans (GAGs), and/or heparin sulfate (HS).

19. The method of claim 1, wherein the administration of the cannabinoids alleviates the neuropathophysiological condition such as dementia, aggressive behavior, hyperactivity, seizure, deafness or loss of vision by at least 10% in comparison with a control that received placebo treatment, without treatment, or the same patient before the cannabinoids treatment.
